(19) United States
(12) Patent Application Publication
(10) Pub. No.: US 2025/0352028 A1
(43) Pub. Date: Nov. 20, 2025

(54) MEDICAL DEVICE, MEDICAL SYSTEM, LEARNING DEVICE, METHOD OF OPERATING MEDICAL DEVICE, AND COMPUTER-READABLE RECORDING MEDIUM

(71) Applicant: OLYMPUS MEDICAL SYSTEMS CORP., Tokyo (JP)

(72) Inventors: Yasuo TANIGAMI, Tokyo (JP); Yusuke OTSUKA, Yokohama-shi (JP); Noriko KURODA, Tokyo (JP); Takaaki IGARASHI, Tokyo (JP)

(73) Assignee: OLYMPUS MEDICAL SYSTEMS CORP., Tokyo (JP)

(21) Appl. No.: 19/290,902

(22) Filed: Aug. 5, 2025

Related U.S. Application Data
(63) Continuation of application No. PCT/JP2023/004455, filed on Feb. 9, 2023.

(51) Int. Cl. A61B 1/00; A61B 1/04

(52) U.S. Cl. CPC A61B 1/000094; A61B 1/000096; A61B 1/0005; A61B 1/043

(57) ABSTRACT

A medical device includes a processor configured to acquire a first image that includes layer information of a biological tissue including a plurality of layers, acquire a second image that is a fluorescence image and includes thermal denaturation information regarding thermal denaturation caused by heat treatment on the biological tissue, acquire correlation information indicating a preset relationship between an emission intensity and a depth of thermal denaturation from a surface layer in the biological tissue, determine a presence or absence of the thermal denaturation of a predetermined layer in the biological tissue based on the first image and the second image, determine a depth of thermal denaturation of the predetermined layer from the surface layer in the biological tissue based on an emission intensity of the fluorescence image and the correlation information, and output depth information regarding the determined depth.

### CROSS REFERENCES TO RELATED APPLICATIONS

This application is a continuation of International Application No. PCT/JP2023/004455, filed on Feb. 9, 2023, the entire contents of which are incorporated herein by reference.

### BACKGROUND

### 1. Technical Field

The present disclosure relates to a medical device, a medical system, a learning device, a method of operating a medical device, and a computer-readable recording medium.

### 2. Related Art

Hitherto, in the medical field, a procedure of transurethrally resecting a bladder tumor (transurethral resection of the bladder tumor (TUR-Bt)) is widely known. In the transurethral resection of the bladder tumor (TUR-Bt), a surgical endoscope (resectoscope) is inserted from the urethra of a subject, and an operator resects a tumor site in a manner of scraping the tumor site from the surface of the tumor site by using a resection treatment tool of an energy device or the like while observing the tumor site of the bladder wall with an eyepiece portion of the surgical endoscope in a state in which the bladder is filled with a perfusate. The bladder wall includes three layers from the inside: a mucosal layer, a muscle layer, and a fat layer. Therefore, a user such as a doctor or an operator performs the transurethral resection of the bladder tumor while identifying the mucosal layer, the muscle layer, and the fat layer.

For example, in WO 2019/244248 A, a biological tissue is irradiated with first light which has a peak wavelength in a first wavelength range including a wavelength at which an absorbance of a biological mucosa is maximized and second light which has a peak wavelength in a second wavelength range including a wavelength at which an absorbance of a muscle layer is maximized, and for which an absorbance of fat is lower than the absorbance of the muscle layer, and an image in which each of the mucosal layer, the muscle layer, and the fat layer can be identified is generated using a first image and a second image obtained by imaging return light from the biological tissue.

### SUMMARY

In some embodiments, a medical device includes a processor including hardware, the processor being configured to acquire a first image that includes layer information of a biological tissue including a plurality of layers, acquire a second image that is a fluorescence image and includes thermal denaturation information regarding thermal denaturation caused by heat treatment on the biological tissue, acquire correlation information indicating a preset relationship between an emission intensity and a depth of thermal denaturation from a surface layer in the biological tissue, determine a presence or absence of the thermal denaturation of a predetermined layer in the biological tissue based on the first image and the second image, determine a depth of thermal denaturation of the predetermined layer from the surface layer in the biological tissue based on an emission intensity of the fluorescence image and the correlation information, and output, as support information, depth information regarding the determined depth of thermal denaturation of the predetermined layer from the surface layer in the biological tissue and information indicating the thermal denaturation of the predetermined layer.

In some embodiments, a medical system includes: a light source device including a first light source configured to generate first light for acquiring layer information of a biological tissue including a plurality of layers, and a second light source configured to generate excitation light that excites advanced glycation end products generated by performing heat treatment on the biological tissue, an imaging device including an imaging element configured to generate an imaging signal by imaging return light or light emitted from the biological tissue irradiated with the first light or the excitation light, and a medical device including a processor including hardware, the processor being configured to generate a first image including the layer information of the biological tissue including the plurality of layers based on the imaging signal generated by imaging the return light with the imaging element, generate a second image that is a fluorescence image and includes thermal denaturation information regarding thermal denaturation caused by the heat treatment on the biological tissue based on the imaging signal generated by imaging the emitted light with the imaging element, acquire correlation information indicating a preset relationship between an emission intensity and a depth of thermal denaturation from a surface layer in the biological tissue, determine a presence or absence of the thermal denaturation of a predetermined layer in the biological tissue based on the first image and the second image, determine a depth of thermal denaturation of the predetermined layer from the surface layer in the biological tissue based on an emission intensity of the fluorescence image and the correlation information, and output, as support information, depth information regarding the determined depth of thermal denaturation of the predetermined layer from the surface layer in the biological tissue and information indicating the thermal denaturation of the predetermined layer.

In some embodiments, a learning device includes a processor including hardware, the processor being configured to generate a trained model by performing machine learning using training data in which a first image that includes layer information of a biological tissue including a plurality of layers and a second image that is a fluorescence image and includes thermal denaturation information regarding thermal denaturation caused by heat treatment on the biological tissue are input data and depth information regarding a depth of thermal denaturation of a predetermined layer from a surface layer in the biological tissue and information indicating the thermal denaturation of the predetermined layer in the biological tissue are output data.

In some embodiments, provided is a method of operating a medical device including a processor. The method includes: acquiring a first image that includes layer information of a biological tissue including a plurality of layers, acquiring a second image that is a fluorescence image and includes thermal denaturation information regarding thermal denaturation caused by heat treatment on the biological tissue, acquiring correlation information indicating a preset relationship between an emission intensity and a depth of thermal denaturation from a surface layer in the biological tissue, determining a presence or absence of the thermal denaturation of a predetermined layer in the biological tissue based on the first image and the second image, determining a depth of thermal denaturation of the predetermined layer from the surface layer in the biological tissue based on an emission intensity of the fluorescence image and the correlation information, and outputting, as support information, depth information regarding the determined depth of thermal denaturation of the predetermined layer from the surface layer in the biological tissue and information indicating the thermal denaturation of the predetermined layer.

In some embodiments, provided is a non-transitory computer-readable recording medium with an executable program stored thereon. The program causes a processor of a medical device to execute: acquiring a first image that includes layer information of a biological tissue including a plurality of layers, acquiring a second image that is a fluorescence image and includes thermal denaturation information regarding thermal denaturation caused by heat treatment on the biological tissue, acquiring correlation information indicating a preset relationship between an emission intensity and a depth of thermal denaturation from a surface layer in the biological tissue, determining a presence or absence of the thermal denaturation of a predetermined layer in the biological tissue based on the first image and the second image, determining a depth of thermal denaturation of the predetermined layer from the surface layer in the biological tissue based on an emission intensity of the fluorescence image and the correlation information, and outputting, as support information, depth information regarding the determined depth of thermal denaturation of the predetermined layer from the surface layer in the biological tissue and information indicating the thermal denaturation of the predetermined layer.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a configuration

of an endoscope system according to a first embodiment;

FIG. 2 is a block diagram illustrating a functional configuration of a main part of the endoscope system according to the first embodiment;

FIG. 3 is a diagram schematically illustrating a wavelength characteristic of light emitted by each of a second light source unit and a third light source unit according to the first embodiment;

FIG. 4 is a diagram schematically illustrating a configuration of a pixel unit according to the first embodiment;

FIG. 5 is a diagram schematically illustrating a configuration of a color filter according to the first embodiment;

FIG. 6 is a diagram schematically illustrating sensitivity and a wavelength band of each filter according to the first embodiment;

FIG. 7A is a diagram schematically illustrating signal values of G pixels of an imaging element according to the first embodiment;

FIG. 7B is a diagram schematically illustrating signal values of R pixels of the imaging element according to the first embodiment;

FIG. 7C is a diagram schematically illustrating signal values of B pixels of the imaging element according to the first embodiment;

FIG. 8 is a diagram schematically illustrating a configuration of a cut filter according to the first embodiment;

FIG. 9 is a diagram schematically illustrating a transmission characteristic of the cut filter according to the first embodiment;

FIG. 10 is a diagram illustrating an example of correlation information recorded by a correlation information recording unit according to the first embodiment;

FIG. 11 is a flowchart illustrating an outline of processing executed by a control device according to the first embodiment;

FIG. 12 is a diagram schematically illustrating a relationship between a hierarchical image and a cross section of a biological tissue;

FIG. 13 is a diagram schematically illustrating a relationship between a thermal denaturation image and a cross section of the biological tissue;

FIG. 14 is a diagram schematically illustrating a relationship between a display image and a cross section of the biological tissue;

FIG. 15 is a diagram illustrating a schematic configuration of an endoscope system according to a second embodiment; and

FIG. 16 is a block diagram illustrating a functional configuration of a medical device according to the second embodiment.

### DETAILED DESCRIPTION

Hereinafter, modes for carrying out the present disclosure will be described in detail with reference to the drawings. Note that the present disclosure is not limited to the following embodiments. In addition, each drawing referred to in the following description merely schematically illustrates a shape, a size, and a positional relationship to the extent that the content of the present disclosure can be understood. That is, the present disclosure is not limited only to the shape, the size, and the positional relationship illustrated in each drawing. Further, in the description of the drawings, the same reference signs denote the same parts. Furthermore, as an example of a medical system according to the present disclosure, an endoscope system including a rigid endoscope and a medical imaging device will be described.

### First Embodiment

### Configuration of Endoscope System

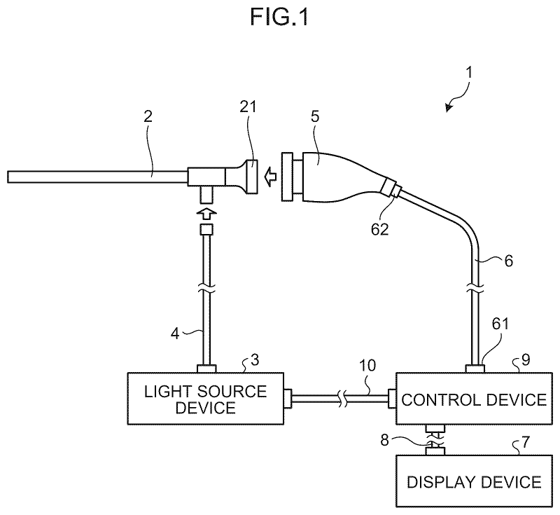

FIG. 1 is a diagram illustrating a configuration of an endoscope system according to a first embodiment. An endoscope system 1 illustrated in FIG. 1 is a system that is used in a medical field and observes and treats a biological tissue in a subject such as a living body. In the first embodiment, a rigid endoscope system using a rigid endoscope (insertion unit 2) illustrated in FIG. 1 will be described as the endoscope system 1, but the present disclosure is not limited thereto, and for example, an endoscope system including a flexible endoscope may be used. Furthermore, an endoscope system can also be applied as the endoscope system 1 to a medical microscope, a medical surgical robot system, or the like that includes a medical imaging device that images a subject and performs surgery, treatment, or the like while displaying an observation image based on an imaging signal (image data) captured by the medical imaging device on a display device. In addition, the endoscope system 1 illustrated in FIG. 1 is used when performing the surgery or treatment on the subject using a treatment tool (not illustrated) of an energy device or the like capable of performing heat treatment. Specifically, the endoscope system 1 illustrated in FIG. 1 is used for transurethral resection of the bladder tumor (TUR-Bt), and is used when performing the treatment on a tumor (bladder cancer) of the bladder or a lesion region.

The endoscope system 1 illustrated in FIG. 1 includes the insertion unit 2, a light source device 3, a light guide 4, an endoscope camera head 5 (endoscope imaging device), a first transmission cable 6, a display device 7, a second transmission cable 8, a control device 9, and a third transmission cable 10.

The insertion unit 2 is rigid or at least partially flexible and has an elongated shape. The insertion unit 2 is inserted into the subject such as a patient via a trocar. The insertion unit 2 is provided with an optical system such as a lens that forms the observation image therein.

The light source device 3 is connected to one end of the light guide 4 and supplies illumination light for irradiating the inside of the subject to one end of the light guide 4 under the control of the control device 9. The light source device 3 is implemented by using one or more light sources of any one of semiconductor laser elements such as a light emitting diode (LED) light source, a xenon lamp, and a laser diode (LD), a processor that is a processing device including hardware such as a field programmable gate array (FPGA) and a central processing unit (CPU), and a memory that is a temporary storage area used by the processor. The light source device 3 and the control device 9 may be configured to perform communication individually as illustrated in FIG. 1, or may be integrated with each other.

The light guide 4 has one end detachably connected to the light source device 3, and the other end detachably connected to the insertion unit 2. The light guide 4 guides the illumination light supplied from the light source device 3 from one end to the other end and supplies the illumination light to the insertion unit 2.

An eyepiece portion 21 of the insertion unit 2 is detachably connected to the endoscope camera head 5. The endoscope camera head 5 generates the imaging signal (RAW data) by receiving the observation image formed by the insertion unit 2 and performing photoelectric conversion, and outputs the imaging signal to the control device 9 via the first transmission cable 6 under the control of the control device 9.

The first transmission cable 6 has one end detachably connected to the control device 9 via a video connector 61, and the other end detachably connected to the endoscope camera head 5 via a camera head connector 62. The first transmission cable 6 transmits the imaging signal output from the endoscope camera head 5 to the control device 9, and transmits setting data, power, and the like output from the control device 9 to the endoscope camera head 5. Here, the setting data is a control signal, a synchronization signal, a clock signal, and the like for controlling the endoscope camera head 5.

The display device 7 displays the observation image based on the imaging signal subjected to image processing in the control device 9 and various types of information regarding the endoscope system 1 under the control of the control device 9. The display device 7 is implemented by using a display monitor such as liquid crystal or organic electro luminescence (EL).

The second transmission cable 8 has one end detachably connected to the display device 7, and the other end detachably connected to the control device 9. The second transmission cable 8 transmits the imaging signal subjected to the image processing in the control device 9 to the display device 7.

The control device 9 is implemented by using a processor that is a processing device including hardware such as a graphics processing unit (GPU), an FPGA, or a CPU, and a memory that is a temporary storage area used by the processor. The control device 9 integrally controls operations of the light source device 3, the endoscope camera head 5, and the display device 7 via each of the first transmission cable 6, the second transmission cable 8, and the third transmission cable 10 according to a program recorded in the memory. In addition, the control device 9 performs various types of image processing on the imaging signal input via the first transmission cable 6 and outputs the imaging signal to the second transmission cable 8. In the first embodiment, the control device 9 functions as a medical device.

The third transmission cable 10 has one end detachably connected to the light source device 3, and the other end detachably connected to the control device 9. The third transmission cable 10 transmits control data from the control device 9 to the light source device 3.

### Functional Configuration of Main Part of Endoscope System

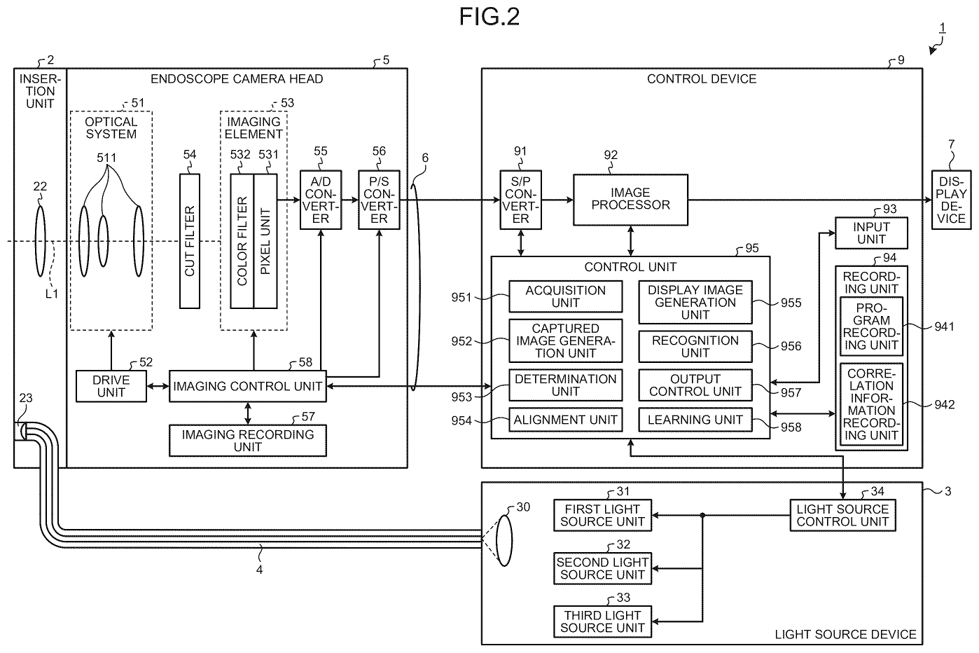

Next, a functional configuration of a main part of the above-described endoscope system 1 will be described. FIG. 2 is a block diagram illustrating the functional configuration of the main part of the endoscope system 1.

### Configuration of Insertion Unit

First, a configuration of the insertion unit 2 will be described. The insertion unit 2 includes an optical system 22 and an illumination optical system 23.

The optical system 22 condenses light such as reflected light reflected from the subject, return light from the subject, excitation light from the subject, and fluorescence emitted from a thermally denatured region thermally denatured by the heat treatment of the energy device or the like to form a subject image. The optical system 22 is implemented by using one or more lenses or the like.

The illumination optical system 23 irradiates the subject with the illumination light supplied from the light guide 4. The illumination optical system 23 is implemented by using one or more lenses or the like.

### Configuration of Light Source Device

Next, a configuration of the light source device 3 will be described. The light source device 3 includes a condenser lens 30, a first light source unit 31, a second light source unit 32, a third light source unit 33, and a light source control unit 34.

The condenser lens 30 condenses light emitted from each of the first light source unit 31, the second light source unit 32, and the third light source unit 33 and emits the light to the light guide 4.

The first light source unit 31 supplies white light (normal light) that is visible light as the illumination light to the light guide 4 by emitting the white light under the control of the light source control unit 34. The first light source unit 31 is implemented using a collimator lens, a white LED lamp, a driver, or the like. The first light source unit 31 may supply the white light that is the visible light by simultaneously performing light emission using a red LED lamp, a green LED lamp, and a blue LED lamp. It is a matter of course that the first light source unit 31 may be implemented using a halogen lamp, a xenon lamp, or the like.

The second light source unit 32 supplies first narrowband light as the illumination light to the light guide 4 by emitting the first narrowband light having a predetermined wavelength band under the control of the light source control unit 34. Here, the first narrowband light has a wavelength band of 530 nm to 550 nm (a central wavelength is 540 nm). The second light source unit 32 is implemented using a green LED lamp, a collimator lens, a transmission filter that transmits light of 530 nm to 550 nm, a driver, or the like.

The third light source unit 33 supplies second narrowband light as the illumination light to the light guide 4 by emitting the second narrowband light having a wavelength band different from that of the first narrowband light under the control of the light source control unit 34. Here, the second narrowband light has a wavelength band of 400 nm to 430 nm (a central wavelength is 415 nm). The third light source unit 33 is implemented by using a semiconductor laser such as a collimator lens or a violet laser diode (LD), a driver, or the like. In the first embodiment, the second narrowband light functions as the excitation light that excites advanced glycation end products generated by performing the heat treatment on the biological tissue.

The light source control unit 34 is implemented by using a processor that is a processing device including hardware such as an FPGA or a CPU, and a memory that is a temporary storage area used by the processor. The light source control unit 34 controls a light emission timing, a light emission time, and the like of each of the first light source unit 31, the second light source unit 32, and the third light source unit 33 based on the control data input from the control device 9.

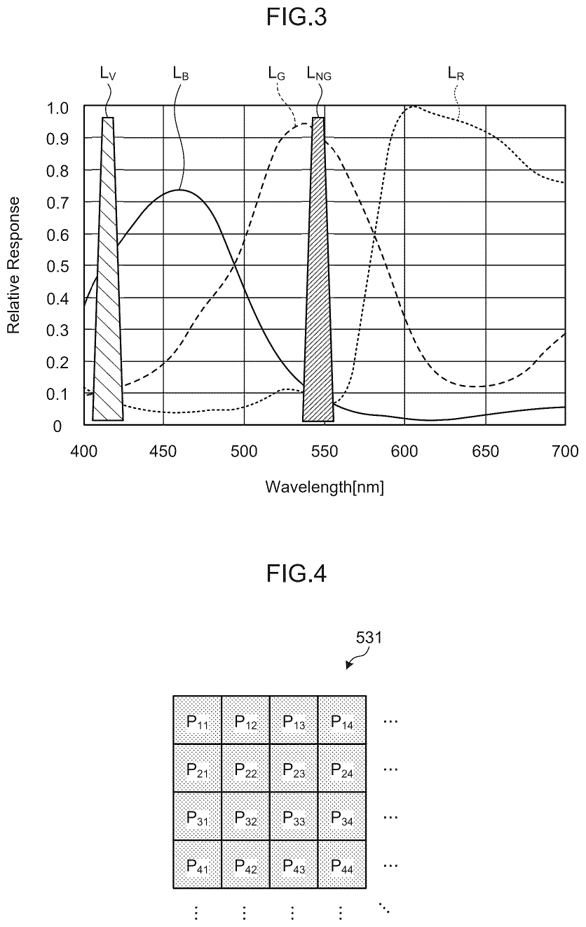

Here, a wavelength characteristic of the light emitted by each of the second light source unit 32 and the third light source unit 33 will be described. FIG. 3 is a diagram schematically illustrating the wavelength characteristic of the light emitted by each of the second light source unit 32 and the third light source unit 33. In FIG. 3, a horizontal axis represents a wavelength (nm), and a vertical axis represents the wavelength characteristic. In FIG. 3, a polygonal line LNG indicates the wavelength characteristic of the first narrowband light emitted by the second light source unit 32, and a polygonal line L_{V} indicates the wavelength characteristic of the second narrowband light (excitation light) emitted by the third light source unit 33. In FIG. 3, a curve L_{B} indicates a blue wavelength band, a curve L_{G} indicates a green wavelength band, and a curve L_{R} indicates a red wavelength band.

As indicated by the polygonal line L_{NG} in FIG. 3, the second light source unit 32 emits the narrowband light having the central wavelength (peak wavelength) of 540 nm and the wavelength band of 530 nm to 550 nm. In addition, the third light source unit 33 emits the excitation light having the central wavelength (peak wavelength) of 415 nm and the wavelength band of 400 nm to 430 nm.

As described above, each of the second light source unit 32 and the third light source unit 33 emits the first narrowband light and the second narrowband light (excitation light) with different wavelength bands.

In addition, the first narrowband light is formed as light for layer discrimination in the biological tissue. Specifically, in the first narrowband light, a difference between an absorbance of a mucosal layer that is the subject and an absorbance of a muscle layer that is the subject is large enough to identify the two subjects. Therefore, in a second image for layer discrimination acquired by irradiation with the first narrowband light for layer discrimination, a region where the imaged mucosal layer appears has a smaller pixel value (luminance value) and is darker than a region where the imaged muscle layer appears. That is, in the first embodiment, it is possible to set a display mode in which the mucosal layer and the muscle layer can be easily identified by using the second image for layer discrimination for generation of a display image.

In addition, the second narrowband light

(excitation light) is light for layer discrimination in the biological tissue and is different from the first narrowband light. Specifically, in the second narrowband light, a difference between the absorbance of the muscle layer that is the subject and an absorbance of a fat layer that is the subject is large enough to identify the two subjects. Therefore, in the second light image for layer discrimination acquired by irradiation with the second narrowband light for layer discrimination, a region where the imaged muscle layer appears has a smaller pixel value (luminance value) and is darker than a region where the imaged fat layer appears. That is, it is possible to set a mode in which the muscle layer and the fat layer are easily identified by using the second image for layer discrimination for generation of the display image.

Both the mucosal layer (biological mucosa) and the muscle layer are the subjects containing a large amount of myoglobin. However, a concentration of myoglobin contained is relatively high in the mucosal layer and relatively low in the muscle layer. A difference in light absorption characteristic between the mucosal layer and the muscle layer is caused by a difference in concentration of myoglobin contained in each of the mucosal layer (biological mucosa) and the muscle layer. The difference in absorbance between the mucosal layer and the muscle layer is maximum in the vicinity of a wavelength at which the absorbance of the biological mucosa has a maximum value. That is, the first narrowband light for layer discrimination is light with which a difference between the mucosal layer and the muscle layer appears larger than light having a peak wavelength in another wavelength band.

In addition, since fat has a lower absorbance for the second narrowband light for layer discrimination than the muscle layer, the pixel value (luminance value) of the region where the imaged muscle layer appears is smaller than the pixel value (luminance value) of the region where the imaged fat layer appears in the second image captured by irradiation with the second narrowband light for layer discrimination. In particular, since the second narrowband light for layer discrimination is light corresponding to a wavelength at which the absorbance of the muscle layer has a maximum value, the second narrowband light is light with which a difference between the muscle layer and the fat layer is large. That is, a difference between the pixel value (luminance value) of a muscle layer region and the pixel value (luminance value) of a fat layer region in the second image for layer discrimination is increased to an identifiable extent.

As described above, the light source device 3 irradiates the biological tissue with each of the first narrowband light and the second narrowband light. As a result, the endoscope camera head 5 described below can obtain an image in which each of the mucosal layer, the muscle layer, and the fat layer included in the biological tissue can be identified by imaging the return light from the biological tissue.

In the first embodiment, the second narrowband light (excitation light) excites the advanced glycation end products generated by performing the heat treatment on the biological tissue by the energy device or the like. In a case where an amino acid and a reducing sugar are heated, a saccharification reaction (Maillard reaction) occurs. The end products resulting from the Maillard reaction are generally called the advanced glycation end products (AGEs). As a characteristic of the AGEs, it is known that a substance having a fluorescence characteristic is contained. That is, in a case where the biological tissue is subjected to the heat treatment by the energy device, the AGEs are generated when the Maillard reaction occurs by heating the amino acid and the reducing sugar in the biological tissue. The AGEs generated by the heating can visualize a state of the heat treatment by fluorescence observation. Furthermore, the AGEs are known to emit stronger fluorescence than an autofluorescent substance originally present in the biological tissue. That is, in the first embodiment, the thermally denatured region obtained by the heat treatment is visualized using the fluorescence characteristic of the AGEs generated in the biological tissue by the heat treatment using the energy device or the like. Therefore, in the first embodiment, the biological tissue is irradiated with the excitation light of blue light having a wavelength of about 415 nm for exciting the AGEs from the second light source unit 32 (excitation light). As a result, in the first embodiment, a fluorescence image (thermal denaturation image) can be observed based on the imaging signal obtained by imaging the fluorescence (for example, green light having a wavelength of 490 to 625 nm) emitted from the thermally denatured region generated from the AGEs.

### Configuration of Endoscope Camera Head

Returning to FIG. 2, the description of the configuration of the endoscope system 1 will be continued.

Next, a configuration of the endoscope camera head 5 will be described. The endoscope camera head 5 includes an optical system 51, a drive unit 52, an imaging element 53, a cut filter 54, an A/D converter 55, a P/S converter 56, an imaging recording unit 57, and an imaging control unit 58.

The optical system 51 forms the subject image condensed by the optical system 22 of the insertion unit 2 on a light receiving surface of the imaging element 53. The optical system 51 can change a focal length and a focal position. The optical system 51 is implemented using a plurality of lenses 511. The optical system 51 changes the focal length and the focal position by moving each of the plurality of lenses 511 on an optical axis L1 by the drive unit 52.

The drive unit 52 moves the plurality of lenses 511 of the optical system 51 along the optical axis L1 under the control of the imaging control unit 58. The drive unit 52 is implemented using a motor such as a stepping motor, a DC motor, or a voice coil motor, and a transmission mechanism such as a gear that transmits rotation of the motor to the optical system 51.

The imaging element 53 is implemented by using a charge coupled device (CCD) or complementary metal oxide semiconductor (CMOS) image sensor including a plurality of pixels arranged in a two-dimensional matrix. The imaging element 53 receives the subject image (light beam) formed by the optical system 51 and passing through the cut filter 54, performs the photoelectric conversion to generate the imaging signal (RAW data), and outputs the imaging signal to the A/D converter 55 under the control of the imaging control unit 58. The imaging element 53 includes a pixel unit 531 and a color filter 532.

FIG. 4 is a diagram schematically illustrating a configuration of the pixel unit 531. As illustrated in FIG. 4, in the pixel unit 531, a plurality of pixels Pₙₘ (n=an integer of 1 or more, and m=an integer of 1 or more) such as photodiodes that accumulate charges according to a light quantity are arranged in a two-dimensional matrix. The pixel unit 531 reads an image signal as the image data from a pixel Pₙₘ in a reading region arbitrarily set as a reading target among the plurality of pixels Pₙₘ, and outputs the image signal to the A/D converter 55 under the control of the imaging control unit 58.

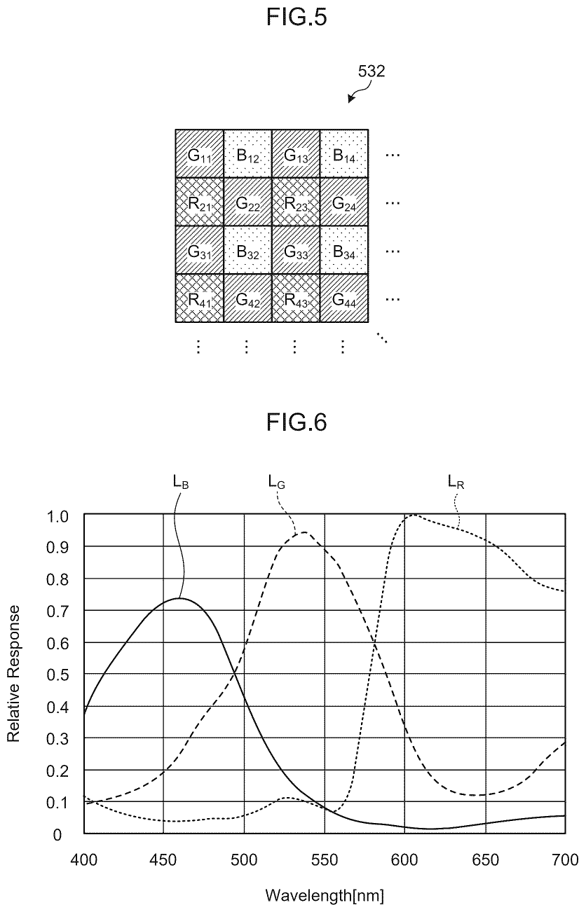

FIG. 5 is a diagram schematically illustrating a configuration of the color filter 532. As illustrated in FIG. 5, the color filter 532 is implemented by a Bayer array having 2×2 as one unit. The color filter 532 is implemented using a filter R that transmits light in the red wavelength band, two filters G that transmit light in the green wavelength band, and a filter B that transmits light in the blue wavelength band.

FIG. 6 is a diagram schematically illustrating sensitivity and a wavelength band of each filter. In FIG. 6, a horizontal axis represents the wavelength (nm), and a vertical axis represents a transmission characteristic (sensitivity characteristic). In FIG. 6, the curve L_{B} indicates the transmission characteristic of the filter B, the curve L_{G} indicates the transmission characteristic of the filter G, and the curve L_{R} indicates the transmission characteristic of the filter R.

As indicated by the curve L_{B} in FIG. 6, the filter B transmits light in the blue wavelength band. As indicated by the curve L_{G} in FIG. 6, the filter G transmits light in the green wavelength band. Further, as indicated by the curve L_{R} in FIG. 6, the filter R transmits light in the red wavelength band. In the following description, a pixel Pₙₘ in which the filter R is disposed on a light receiving surface will be described as an R pixel, a pixel Pₙₘ in which the filter G is disposed on a light receiving surface will be described as a G pixel, and a pixel Pₙₘ in which the filter B is disposed on a light receiving surface will be described as a B pixel.

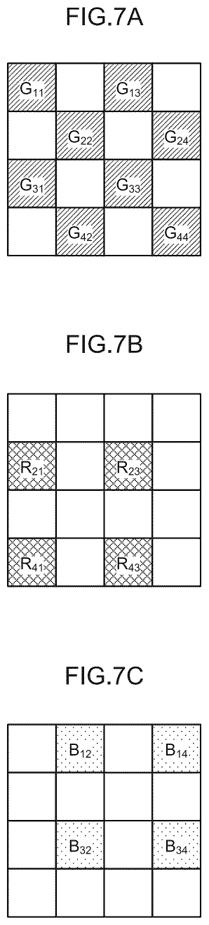

With the imaging element 53 configured as described above, in a case where the subject image formed by the optical system 51 is received, as illustrated in FIGS. 7A to 7C, color signals (an R signal, a G signal, and a B signal) of the R pixel, the G pixel, and the B pixel are generated.

Returning to FIG. 2, the description of the configuration of the endoscope system 1 will be continued.

The cut filter 54 is disposed on the optical axis L1 between the optical system 51 and the imaging element 53. The cut filter 54 is provided on a light receiving surface side (incident surface side) of the G pixel provided with the filter G that transmits at least the green wavelength band of the color filter 532. The cut filter 54 blocks light in a shorter wavelength band including the wavelength band of the excitation light, and transmits a longer wavelength band beyond the wavelength band of the excitation light.

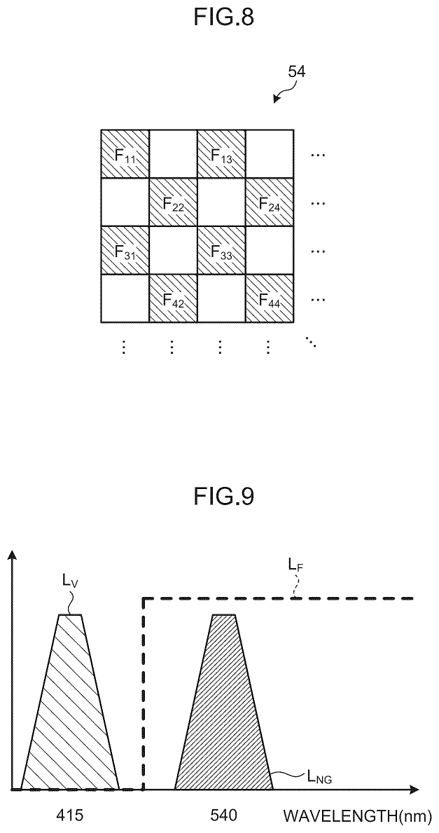

FIG. 8 is a diagram schematically illustrating a configuration of the cut filter 54. As illustrated in FIG. 8, a filter F₁₁ included in the cut filter 54 is disposed at a position where a filter G₁₁ (see FIG. 5) is disposed, on the light receiving surface side immediately above the filter G₁₁.

FIG. 9 is a diagram schematically illustrating a transmission characteristic of the cut filter 54. In FIG. 8, a horizontal axis represents the wavelength (nm), and a vertical axis represents the transmission characteristic. In FIG. 8, a polygonal line L_{F} indicates the transmission characteristic of the cut filter 54, the polygonal line L_{NG} indicates the wavelength characteristic of the first narrowband light, and the polygonal line L_{V} indicates the wavelength characteristic of the second narrowband light (excitation light).

As illustrated in FIG. 9, the cut filter 54 blocks the wavelength band of the second narrowband light (excitation light) and transmits the longer wavelength band beyond the wavelength band of the second narrowband light (excitation light). Specifically, the cut filter 54 blocks the light in the shorter wavelength band of 400 nm to less than 430 nm including the wavelength band of the second narrowband light (excitation light), and transmits the light in the longer wavelength band beyond the wavelength band of 400 nm to 430 nm including the second narrowband light (excitation light).

Returning to FIG. 2, the description of the configuration of the endoscope camera head 5 will be continued.

The A/D converter 55 executes A/D conversion processing on the analog imaging signal input from the imaging element 53, and outputs the analog imaging signal to the P/S converter 56 under the control of the imaging control unit 58. The A/D converter 55 is implemented by using an A/D conversion circuit or the like.

The P/S converter 56 performs parallel/serial conversion on the digital imaging signal input from the A/D converter 55, and outputs the imaging signal subjected to the parallel/serial conversion to the control device 9 via the first transmission cable 6 under the control of the imaging control unit 58. The P/S converter 56 is implemented by using a P/S conversion circuit or the like. In the first embodiment, an E/O converter that converts the imaging signal into an optical signal may be provided instead of the P/S converter 56, and the imaging signal may be output to the control device 9 by the optical signal, or the imaging signal may be transmitted to the control device 9 by wireless communication such as Wireless Fidelity (Wi-Fi) (registered trademark).

The imaging recording unit 57 records various types of information (for example, pixel information of the imaging element 53 and the characteristic of the cut filter 54) regarding the endoscope camera head 5. Furthermore, the imaging recording unit 57 records various types of setting data and control parameters transmitted from the control device 9 via the first transmission cable 6. The imaging recording unit 57 is implemented using a nonvolatile memory or a volatile memory.

The imaging control unit 58 controls an operation of each of the drive unit 52, the imaging element 53, the A/D converter 55, and the P/S converter 56 based on the setting data received from the control device 9 via the first transmission cable 6. The imaging control unit 58 is implemented by using a timing generator (TG), a processor including hardware such as an application specific integrated circuit (ASIC) or a CPU, and a memory that is a temporary storage area used by the processor.

### Configuration of Control Device

Next, a configuration of the control device 9 will be described.

The control device 9 includes an S/P converter 91, an image processor 92, an input unit 93, a recording unit 94, and a control unit 95.

The S/P converter 91 performs serial/parallel conversion on the image data received from the endoscope camera head 5 via the first transmission cable 6 and outputs the image data to the image processor 92 under the control of the control unit 95. In a case where the endoscope camera head 5 outputs the imaging signal as the optical signal, an O/E converter that converts the optical signal into an electric signal may be provided instead of the S/P converter 91. Furthermore, in a case where the endoscope camera head 5 transmits the imaging signal by wireless communication, a communication module capable of receiving a wireless signal may be provided instead of the S/P converter 91.

The image processor 92 executes predetermined image processing on the imaging signal of the parallel data input from the S/P converter 91 and outputs the imaging signal to the display device 7 under the control of the control unit 95. Here, the predetermined image processing is demosaic processing, white balance processing, gain adjustment processing, y correction processing, format conversion processing, or the like. The image processor 92 is implemented by using a processor that is a processing device including hardware such as a GPU or an FPGA and a memory that is a temporary storage area used by the processor.

Furthermore, in a case where the light source device 3 performs irradiation with white light toward the biological tissue, the image processor 92 executes the image processing on the imaging signal input from the endoscope camera head 5 via the S/P converter 91 to generate a white light image. Furthermore, in a case where the light source device 3 performs irradiation with the first narrowband light and the second narrowband light, the image processor 92 executes the image processing on signal values of the G pixel and the B pixel included in the imaging signal input from the endoscope camera head 5 via the S/P converter 91 to generate a pseudo color image (narrowband image). In this case, the signal value of the G pixel includes deep mucosal layer information of the subject. Furthermore, the signal value of the B pixel includes surface mucosal layer information of the subject. Therefore, the image processor 92 executes the image processing such as gain control processing, pixel interpolation processing, and mucosal enhancement processing on the signal value of each of the G pixel and the B pixel included in the imaging signal to generate the pseudo color image, and outputs the pseudo color image to the display device 7. Here, the pseudo color image is an image generated using only the signal value of the G pixel and the signal value of the B pixel. The image processor 92 acquires a signal value of the R pixel, but does not use the signal value for generating the pseudo color image and deletes the signal value.

Furthermore, in a case where the light source device 3 performs irradiation with the second narrowband light (excitation light), the image processor 92 is included in the imaging signal input from the endoscope camera head 5 via the S/P converter 91. The image processing is executed on the signal value of each of the G pixel and the B pixel to generate the fluorescence image (pseudo color image). In this case, the signal value of the G pixel includes fluorescence information emitted from a heat treatment region. Furthermore, the B pixel includes background information regarding a biological tissue around the heat treatment region. Therefore, the image processor 92 executes the image processing such as the gain control processing, the pixel interpolation processing, and the mucosal enhancement processing on the signal value of each of the G pixel and the B pixel included in the image data to generate the fluorescence image (pseudo color image), and outputs the fluorescence image (pseudo color image) to the display device 7. In this case, the image processor 92 executes the gain control processing of making a gain for the signal value of the G pixel larger than a gain for the signal value of the G pixel at the time of normal light observation, and making a gain for the signal value of the B pixel smaller than a gain for the signal value of the B pixel at the time of the normal light observation. Furthermore, the image processor 92 executes the gain control processing such that the signal value of the G pixel and the signal value of the B pixel are the same as each other (1:1).

The input unit 93 receives various operations related to the endoscope system 1 and outputs the received operations to the control unit 95. The input unit 93 is implemented using a mouse, a foot switch, a keyboard, a button, a switch, a touch panel, or the like.

The recording unit 94 is implemented by using a recording medium such as a volatile memory, a nonvolatile memory, a solid state drive (SSD), a hard disk drive (HDD), or a memory card. The recording unit 94 records data including various parameters and the like necessary for an operation of the endoscope system 1. In addition, the recording unit 94 includes a program recording unit 941 that records various programs for operating the endoscope system 1 and a correlation information recording unit 942 that records correlation information indicating a correlation between the degree of invasion (depth) into the biological tissue by the heat treatment and an emission intensity. Details of the correlation information will be described below.

The control unit 95 is implemented by using a processor including hardware such as an FPGA or a CPU, and a memory that is a temporary storage area used by the processor. The control unit 95 integrally controls each unit included in the endoscope system 1. Specifically, the control unit 95 reads and executes the program recorded in the program recording unit 941 in a work area of the memory, and controls each component and the like through the execution of the program by the processor, so that the hardware and software cooperate with each other to implement a functional module matching a predetermined purpose. Specifically, the control unit 95 includes an acquisition unit 951, a captured image generation unit 952, a determination unit 953, an alignment unit 954, a display image generation unit 955, a recognition unit 956, an output control unit 957, and a learning unit 958.

The acquisition unit 951 acquires the imaging signal generated by the endoscope camera head 5 via the S/P converter 91 and the image processor 92. Specifically, the acquisition unit 951 acquires the imaging signal of the white light generated by the endoscope camera head 5 when the light source device 3 irradiates the biological tissue with the white light, a first image signal generated by the endoscope camera head 5 when the light source device 3 irradiates the biological tissue with the first narrowband light and the second narrowband light, and a second image signal generated by the endoscope camera head 5 when the light source device 3 irradiates the biological tissue with the second narrowband light (excitation light).

The captured image generation unit 952 generates a hierarchical image in which the mucosal layer, the muscle layer, and the fat layer in the biological tissue can be identified for each layer based on the first image signal and the second image signal acquired by the acquisition unit 951. The captured image generation unit 952 generates the thermal denaturation image based on a third image signal acquired by the acquisition unit 951. Furthermore, the captured image generation unit 952 generates the white light image based on a white light image signal acquired by the acquisition unit 951.

The determination unit 953 determines the depth of thermal denaturation based on the correlation information recorded by the correlation information recording unit 942 and a fluorescence intensity from the thermally denatured region included in a thermal denaturation image P2. Here, the depth is a length from the surface (surface layer) of the biological tissue toward the fat layer.

The alignment unit 954 executes alignment processing of the hierarchical image and the thermal denaturation image generated by the captured image generation unit 952.

The display image generation unit 955 generates the display image by combining the hierarchical image and the thermal denaturation image subjected to the alignment treatment by the alignment unit 954. Specifically, the alignment unit 954 executes the alignment processing of the hierarchical image and the thermal denaturation image based on a position where feature data of each pixel forming the hierarchical image is aligned with feature data of each pixel forming the thermal denaturation image. Here, the feature data is, for example, the pixel value, the luminance value, the edge, or the contrast. The display image generation unit 955 may generate the display image by superimposing a different display mode for each layer of the biological tissue in which the thermal denaturation has occurred on the white light image generated by the captured image generation unit 952 based on the hierarchical image that is a first image and the thermal denaturation image that is a second image. Furthermore, the display image generation unit 955 may generate the display image in which the thermal denaturation of a layer selected by a user according to an instruction signal input from the input unit 93 among the layers of the biological tissue in which the thermal denaturation has occurred is highlighted based on the hierarchical image that is the first image and the thermal denaturation image that is the second image.

The recognition unit 956 determines the presence or absence of the thermal denaturation of a predetermined layer in the biological tissue based on the hierarchical image that is the first image and the thermal denaturation image that is the second image. Specifically, the recognition unit 956 individually recognizes the thermal denaturation of each of the mucosal layer, the muscle layer, and the fat layer included in the biological tissue included in the display image generated by the display image generation unit 955 based on the depth of thermal denaturation determined by the determination unit 953.

The output control unit 957 outputs support information indicating the thermal denaturation of the predetermined layer based on a result (recognition result) of determining the presence or absence of the thermal denaturation by the recognition unit 956. Specifically, the output control unit 957 outputs the different display mode of the thermal denaturation for each layer to the display device 7 based on the display image generated by the display image generation unit 955 and a result of recognizing the thermal denaturation of each layer by the recognition unit 956. Furthermore, the output control unit 957 may change a type of the display image generated by the display image generation unit 955 based on the instruction signal input from the input unit 93 and output the display image to the display device 7. For example, based on the instruction signal input from the input unit 93, the output control unit 957 outputs, to the display device 7, the display image generated by superimposing the different display mode for each layer of the biological tissue in which the thermal denaturation has occurred on the white light image generated by the captured image generation unit 952 and the display image in which the thermal denaturation of the layer selected by the user is highlighted.

The learning unit 958 generates a trained model by performing machine learning using training data in which the hierarchical image that is the first image including layer information of the biological tissue including a plurality of layers and the thermal denaturation image that is the second image including thermal denaturation information regarding the thermal denaturation of the biological tissue by the heat treatment are input data, and the support information indicating the thermal denaturation of the predetermined layer in the biological tissue is output data. Specifically, the learning unit 958 may generate the trained model by performing machine learning using the training data in which the fluorescence image obtained by irradiating the biological tissue with the excitation light and imaging the fluorescence and the white light image obtained by performing imaging by irradiating the biological tissue with the white light are the input data and the support information indicating the thermal denaturation of the predetermined layer in the biological tissue is the output data. Here, the trained model includes a neural network in which each layer includes one or more nodes.

Furthermore, a type of the machine learning is not particularly limited, and for example, it is sufficient if the training data and learning data in which a plurality of hierarchical images and a plurality of thermal denaturation images are associated with the depth of thermal denaturation or the recognition result for the thermal denaturation related to the thermal denaturation by the heat treatment, which is recognized from the hierarchical images and the plurality of thermal denaturation images or to which or an annotation is applied, are prepared, and learning is performed by inputting the training data and the learning data to a calculation model based on a multilayer neural network.

Furthermore, as a method for the machine learning, for example, a method based on a deep neural network (DNN) of the multilayer neural network such as a convolutional neural network (CNN) or a 3D-CNN is used.

Furthermore, as the method for the machine learning, a method based on a recurrent neural network (RNN), a long short-term memory unit (LSTM) obtained by extending the RNN, or the like may be used. A control unit of a learning device different from the control device 9 may execute such functions to generate the trained model. It is a matter of course that the function of the learning unit 958 may be provided in the image processor 92.

### Details of Correlation Information

Next, an example of the correlation information recorded by the correlation information recording unit 942 will be described.

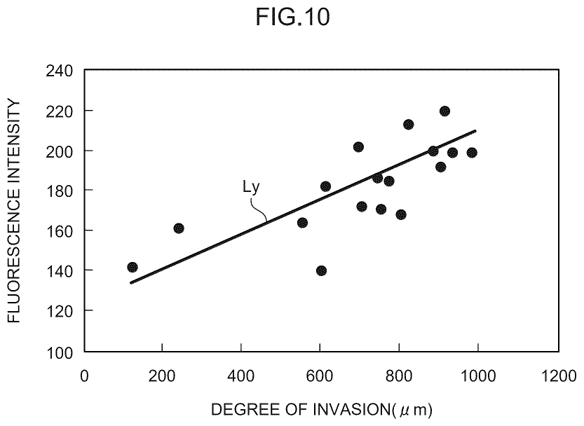

FIG. 10 is a diagram illustrating an example of the correlation information recorded by the correlation information recording unit 942. In FIG. 10, a vertical axis represents the emission intensity, and a horizontal axis represents the degree of invasion (depth and region) into the biological tissue by the heat treatment. In FIG. 10, a straight line Ly indicates a correlation between the emission intensity and the degree of invasion (depth and region) into the biological tissue by the heat treatment.

As indicated by the straight line Ly in FIG. 10, the emission intensity increases as the degree of invasion into the biological tissue by the heat treatment increases.

### Processing in Control Device

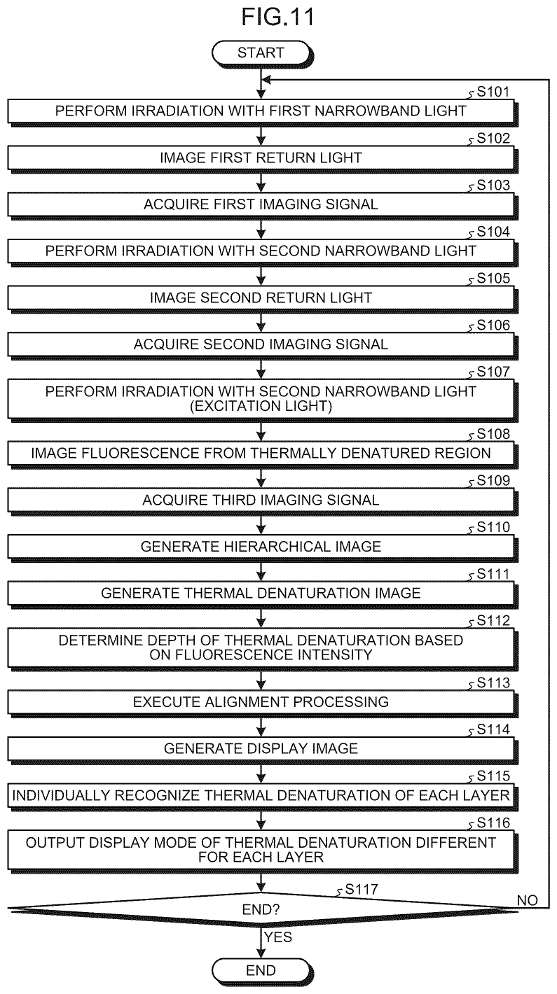

Next, processing executed by the control device 9 will be described. FIG. 11 is a flowchart illustrating an outline of the processing executed by the control device 9.

As illustrated in FIG. 11, first, the control unit 95 controls the light source control unit 34 of the light source device 3 to cause the second light source unit 32 to emit light and supply the first narrowband light to the insertion unit 2, thereby irradiating the biological tissue with the first narrowband light (step S101).

Subsequently, the control unit 95 controls the imaging control unit 58 to cause the imaging element 53 to image first return light from the biological tissue (step S102).

Thereafter, the acquisition unit 951 acquires the first imaging signal generated by imaging performed by the imaging element 53 of the endoscope camera head 5 (step S103).

Subsequently, the control unit 95 controls the light source control unit 34 of the light source device 3 to cause the second light source unit 32 to emit light and supply the second narrowband light to the insertion unit 2, thereby irradiating the biological tissue with the second narrowband light (step S104).

Subsequently, the control unit 95 controls the imaging control unit 58 to cause the imaging element 53 to image second return light from the biological tissue (step S105).

Thereafter, the acquisition unit 951 acquires a second imaging signal generated by imaging performed by the imaging element 53 of the endoscope camera head 5 (step S106).

Subsequently, the control unit 95 controls the light source control unit 34 of the light source device 3 to cause the third light source unit 33 to emit light and supply the second narrowband light that is the excitation light to the insertion unit 2, thereby irradiating the biological tissue with the excitation light (step S107).

Subsequently, the control unit 95 controls the imaging control unit 58 to cause the imaging element 53 to image the fluorescence from the thermally denatured region of the biological tissue (step S108).

Thereafter, the acquisition unit 951 acquires a third imaging signal generated by imaging performed by the imaging element 53 of the endoscope camera head 5 (step S109).

Subsequently, the captured image generation unit 952 generates the hierarchical image in which the mucosal layer, the muscle layer, and the fat layer in the biological tissue can be identified for each layer based on the first image signal and the second image signal acquired by the acquisition unit 951 (step S110). After step S110, the control device 9 proceeds to step S111 described below.

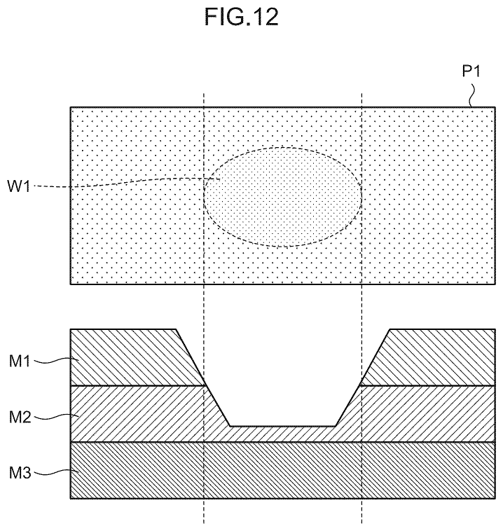

FIG. 12 is a diagram schematically illustrating a relationship between the hierarchical image and a cross section of the biological tissue. In FIG. 12, the upper part illustrates a hierarchical image Pl, and the lower part illustrates each layer of the biological tissue. As illustrated in FIG. 12, the hierarchical image P1 includes a muscle layer exposure region W1 exposed to a mucosal layer M1 and a muscle layer M2 by the heat treatment using the energy device or the like. That is, the hierarchical image P1 is in a state of not reaching a fat layer M3 by the heat treatment using the energy device or the like.

Returning to FIG. 11, the description of step S111 and subsequent steps will be continued.

In step S111, the captured image generation unit 952 generates the thermal denaturation image based on the third image signal acquired by the acquisition unit 951. After step S112, the control device 9 proceeds to step S112 described below.

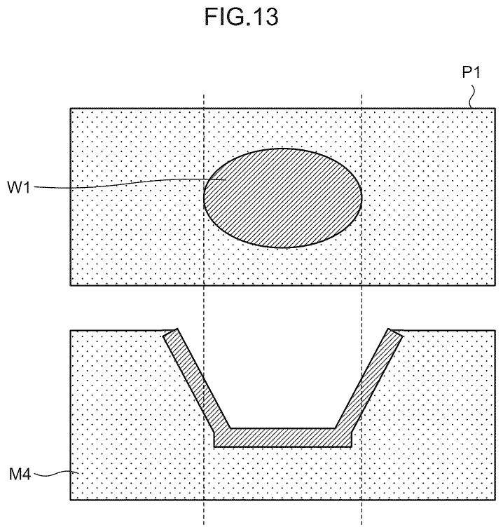

FIG. 13 is a diagram schematically illustrating a relationship between the thermal denaturation image and a cross section of the biological tissue. In FIG. 13, the upper part illustrates the thermal denaturation image P2, and the lower part illustrates each layer of the biological tissue. As illustrated in FIG. 13, the thermal denaturation image P2 includes a thermally denatured region W2 generated by the heat treatment using the energy device or the like.

Returning to FIG. 11, the description of step S112 and subsequent steps will be continued.

In step S112, the determination unit 953 determines the depth of thermal denaturation based on the correlation information recorded by the correlation information recording unit 942 and the fluorescence intensity from the thermally denatured region included in the thermal denaturation image P2. Here, the depth is a length from the surface of the biological tissue toward the fat layer.

Thereafter, the alignment unit 954 executes the alignment processing of the hierarchical image P1 and the thermal denaturation image P2 (step S113). Specifically, the alignment unit 954 executes the alignment processing such that a position of the feature data included in the hierarchical image P1 and a position of the feature data included in the thermal denaturation image P2 are aligned with each other by using a known technology. For example, the alignment unit 954 executes the alignment processing of the hierarchical image P1 and the thermal denaturation image P2 based on a position where the feature data of each pixel forming the hierarchical image P1 is aligned with the feature data of each pixel forming the thermal denaturation image P2. Here, the feature data is, for example, the pixel value, the luminance value, the edge, or the contrast.

Subsequently, the display image generation unit 955 generates the display image by combining the hierarchical image P1 and the thermal denaturation image P2 subjected to the alignment treatment by the alignment unit 954 (step S114).

Thereafter, the recognition unit 956 individually recognizes the thermal denaturation of each of the mucosal layer, the muscle layer, and the fat layer included in the biological tissue included in the display image generated by executing the alignment processing by the alignment unit 954 based on the depth of thermal denaturation determined by the determination unit 953 (step S115). Specifically, the recognition unit 956 individually recognizes the thermal denaturation of each of the mucosal layer M1, the muscle layer M2, and the fat layer M3 included in a display image P3 based on the depth of thermal denaturation determined by the determination unit 953. In this case, the recognition unit 956 individually recognizes the thermal denaturation of each of the mucosal layer M1, the muscle layer M2, and the fat layer M3 included in the display image P3 based on the depth of thermal denaturation determined by the determination unit 953.

Subsequently, the output control unit 957 outputs the different display mode of the thermal denaturation for each layer to the display device 7 based on the display image generated by the display image generation unit 955 and the result of recognizing the thermal denaturation of each layer by the recognition unit 956 (step S116).

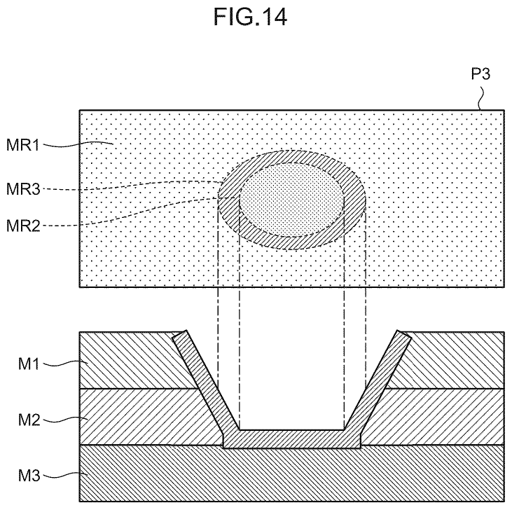

FIG. 14 is a diagram schematically illustrating a relationship between the display image and a cross section of the biological tissue. In FIG. 14, the upper part illustrates the display image P3, and the lower part illustrates each layer of the biological tissue. As illustrated in FIG. 14, the output control unit 957 outputs the display image P3 with the different display mode of the thermal denaturation for each layer as the support information to the display device 7 based on the display image generated by the display image generation unit 955 and the result of recognizing the thermal denaturation of each layer by the recognition unit 956. Specifically, the output control unit 957 displays display areas respectively corresponding to the mucosal layer M1, the muscle layer M2, and the fat layer M3 in “yellow”, “green”, and “blue”. For example, in FIG. 14, the output control unit 957 outputs, to the display device 7, a thermally denatured region MR2 of the muscle layer and a thermally denatured region MR3 of the fat layer in the display image P3 such that the thermally denatured region of the muscle layer and the thermally denatured region of the fat layer are displayed in distinguishable colors, for example, the thermally denatured region of the muscle layer is displayed in “green”, and the thermally denatured region of the fat layer is displayed in “blue”. As a result, the user can intuitively grasp the presence or absence of thermal denaturation of a layer that is not exposed on the surface. In FIG. 14, the output control unit 957 can identify the display area corresponding to each of the mucosal layer M1, the muscle layer M2, and the fat layer M3 by different colors. However, the present disclosure is not limited thereto. For example, the output control unit 957 may output the display area to the display device 7 so as to highlight a contour of the display area corresponding to each of the mucosal layer, the muscle layer, and the fat layer for each depth of thermal degeneration. It is a matter of course that the output control unit 957 may superimpose the depth of thermal denaturation determined by the determination unit 953 on the display image P3 and output the display image P3 on which the depth of thermal denaturation is superimposed to the display device 7 as the support information.

Subsequently, the control unit 95 determines whether or not an end signal for ending the observation of the subject by the endoscope system 1 has been input from the input unit 93 (step S117). In a case where the control unit 95 determines that the end signal for ending the observation of the subject by the endoscope system 1 has been input from the input unit 93 (step S117: Yes), the control device 9 ends the processing. On the other hand, in a case where the control unit 95 determines that the end signal for ending the observation of the subject by the endoscope system 1 has not been input from the input unit 93 (step S117: No), the control device 9 returns to step S101 described above.

According to the first embodiment described above, the output control unit 957 outputs the display image P3 with the display mode of the thermal denaturation different for each layer to the display device 7 as the support information based on the presence or absence of the thermal denaturation of each layer of the biological tissue recognized by the recognition unit 956. As a result, the user can confirm the depth of thermal denaturation into the biological tissue.

Further, according to the first embodiment, the output control unit 957 may output, to the display device 7, the different display mode for each layer depending on the depth of thermal denaturation based on the display image P3 generated by the display image generation unit 955 and the result of recognizing the thermal denaturation of each layer by the recognition unit 956.

In addition, according to the first embodiment, the recognition unit 956 individually recognizes (determines) the thermal denaturation of each of the mucosal layer, the muscle layer, and the fat layer included in the biological tissue included in the display image generated by executing the alignment processing by the alignment unit 954 based on the depth of thermal denaturation determined by the determination unit 953, and the output control unit 957 outputs the display image P3 with the display mode to the display device 7 according to the result of recognizing the thermal denaturation of each layer by the recognition unit 956. As a result, the user can grasp the presence or absence of the thermal denaturation of each of the mucosal layer, the muscle layer, and the fat layer.

In the first embodiment, the output control unit 957 may output depth information regarding the depth of thermal denaturation determined by the determination unit 953 as the support information.

Furthermore, in the first embodiment, the learning unit 958 is provided in the control device 9, but the present disclosure is not limited thereto, and the learning unit 958 that generates the trained model may be provided in a device different from the control device 9, such as a learning device or a server connectable via a network.

Furthermore, in the first embodiment, the output control unit 957 may output, to the display device 7, the display image generated by superimposing the display mode different for each layer of the biological tissue in which the thermal denaturation has occurred on the white light image generated by the captured image generation unit 952, the display image being generated by the display image generation unit 955. As a result, the user can grasp the presence or absence of the thermal denaturation of each of the mucosal layer, the muscle layer, and the fat layer.

In addition, in the first embodiment, the display image generation unit 955 may generate the display image in which the thermal denaturation of the layer selected by the user according to the instruction signal input from the input unit 93 among the layers of the biological tissue in which the thermal denaturation has occurred is highlighted, based on the hierarchical image that is the first image and the thermal denaturation image that is the second image, and the output control unit 957 may output the display image generated by the display image generation unit 955 to the display device 7. As a result, the user can confirm thermal denaturation of a desired layer.

### Second Embodiment

Next, a second embodiment will be described. In the first embodiment described above, the control unit 95 of the control device 9 determines the presence or absence of the thermal denaturation of the predetermined layer in the biological tissue based on the hierarchical image that is the first image including the layer information of the biological tissue including the plurality of layers and the thermal denaturation image that is the second image including the thermal denaturation information, and outputs the support information indicating the thermal denaturation of the predetermined layer to the display device 7. However, in the second embodiment, a medical device that outputs support information is separately provided. Hereinafter, a configuration of an endoscope system according to the second embodiment will be described. Note that the same components as those of the endoscope system 1 according to the first embodiment described above are denoted by the same reference signs, and a detailed description thereof will be omitted.

### Configuration of Endoscope System

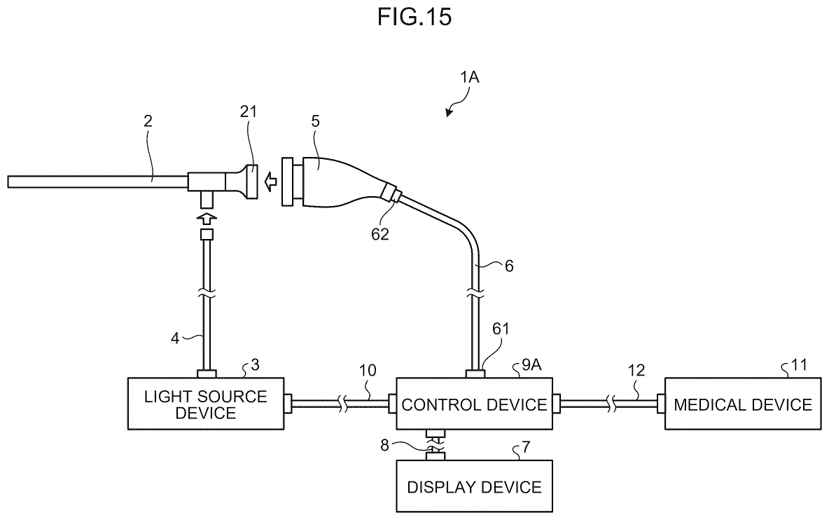

FIG. 15 is a diagram illustrating a schematic configuration of the endoscope system according to the second embodiment. An endoscope system 1A illustrated in FIG. 15 includes a control device 9A instead of the control device 9 of the endoscope system 1 according to the first embodiment described above. Furthermore, the endoscope system 1A further includes a medical device 11 and a fourth transmission cable 12 in addition to the configuration of the endoscope system 1 according to the first embodiment described above.

The control device 9A is implemented by using a processor that is a processing device including hardware such as a GPU, an FPGA, or a CPU, and a memory that is a temporary storage area used by the processor. The control device 9A integrally controls operations of a light source device 3, an endoscope camera head 5, a display device 7, and the medical device 11 via each of a first transmission cable 6, a second transmission cable 8, a third transmission cable 10, and the fourth transmission cable 12 according to a program recorded in the memory. The control device 9A is different from the control unit 95 according to the first embodiment described above in that functions of an acquisition unit 951, a captured image generation unit 952, a determination unit 953, an alignment unit 954, a display image generation unit 955, a recognition unit 956, an output control unit 957, and a learning unit 958 are not provided.

The medical device 11 is implemented by using a processor that is a processing device including hardware such as a GPU, an FPGA, or a CPU, and a memory that is a temporary storage area used by the processor. The medical device 11 acquires various types of information from the control device 9A via the fourth transmission cable 12, and outputs the acquired various types of information to the control device 9A. Note that a detailed functional configuration of the medical device 11 will be described below.

The fourth transmission cable 12 has one end detachably connected to the control device 9A, and the other end detachably connected to the medical device 11. The fourth transmission cable 12 transmits various types of information from the control device 9A to the medical device 11, and transmits various types of information from the medical device 11 to the control device 9A.

### Functional Configuration of Medical Device

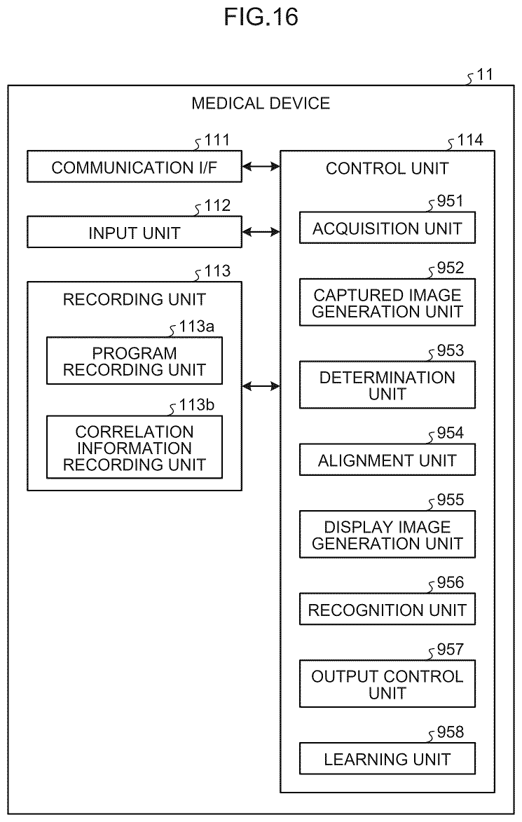

FIG. 16 is a block diagram illustrating a functional configuration of the medical device 11. The medical device 11 illustrated in FIG. 16 includes a communication interface (I/F) 111, an input unit 112, a recording unit 113, and a control unit 114.

The communication I/F 111 is an interface for communicating with the control device 9A via the fourth transmission cable 12. The communication I/F 111 receives various types of information from the control device 9A according to a predetermined communication standard, and outputs the received various types of information to the control unit 114.

The input unit 112 receives inputs of various operations related to the endoscope system 1A and outputs the received operations to the control unit 114. The input unit 112 is implemented using a mouse, a foot switch, a keyboard, a button, a switch, a touch panel, and the like.

The recording unit 113 is implemented by using a recording medium such as a volatile memory, a nonvolatile memory, an SSD, an HDD, or a memory card. The recording unit 113 records data including various parameters and the like necessary for an operation of the medical device 11. In addition, the recording unit 113 includes a program recording unit 113a that records various programs for operating the medical device 11, and a correlation information recording unit 113b that records correlation information indicating a correlation between the degree of invasion (depth) into a biological tissue by heat treatment and an emission intensity.

The control unit 114 is implemented by using a processor including hardware such as an FPGA or a CPU, and a memory that is a temporary storage area used by the processor. The control unit 114 integrally controls each unit included in the medical device 11. The control unit 114 has the same function as the control unit 95 according to the first embodiment described above. Specifically, the control unit 114 includes an acquisition unit 951, a captured image generation unit 952, a determination unit 953, an alignment unit 954, a display image generation unit 955, a recognition unit 956, an output control unit 957, and a learning unit 958.

The medical device 11 configured as described above executes processing similar to that of the control device 9 according to the first embodiment described above, and outputs a result of the processing to the control device 9A. In this case, the control device 9A outputs, to the display device 7, a different display mode for each layer depending on the depth of thermal denaturation based on a result of recognizing the thermal denaturation of each layer by the recognition unit 956 in a display image generated by an image processor 92 based on a processing result of the medical device 11 to display each layer.

According to the second embodiment described above, it is possible to obtain an effect similar to that of the first embodiment described above, that is, the user can confirm the depth of thermal denaturation into the biological tissue.

### Other Embodiments

Various embodiments can be formed by appropriately combining a plurality of constituent elements disclosed in the endoscope systems according to the first and second embodiments of the present disclosure described above. For example, some constituent elements may be deleted from all the constituent elements described in the endoscope systems according to the embodiments of the present disclosure described above. Furthermore, the constituent elements described in the endoscope systems according to the embodiments of the present disclosure described above may be appropriately combined.

Furthermore, in the endoscope systems according to the first and second embodiments of the present disclosure, the constituent elements are connected to each other in a wired manner, or may be connected wirelessly via a network.

Furthermore, in the first and second embodiments of the present disclosure, the function of the control unit included in the endoscope system, and the functional modules of the acquisition unit 951, the captured image generation unit 952, the determination unit 953, the alignment unit 954, the display image generation unit 955, the recognition unit 956, and the output control unit 957 may be provided in a server or the like connectable via a network. It is a matter of course that a server may be provided for each functional module.

In addition, in the first and second embodiments of the present disclosure, an example in which the endoscope system is used for the transurethral resection of the bladder tumor has been described, but the present disclosure is not limited thereto, and the endoscope system can be applied to various medical procedures for resection of a lesion by, for example, the energy device or the like.

Furthermore, in the endoscope systems according to the first and second embodiments of the present disclosure, the “unit” described above can be replaced with “means”, “circuit”, or the like. For example, the control unit can be replaced with control means or a control circuit.

Note that, in the description of the flowcharts in the present specification, the context of processing between steps is clearly indicated using expressions such as “first”, “thereafter”, and “subsequently”, but the order of processing necessary for implementing the embodiments is not uniquely determined by these expressions. That is, the order of processing in the flowcharts described in the present specification can be changed within a range without inconsistency.

According to the present disclosure, it is possible to confirm a depth of thermal denaturation into a biological tissue.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical device comprising a processor comprising hardware, the processor being configured to
acquire a first image that includes layer information of a biological tissue including a plurality of layers,
acquire a second image that is a fluorescence image and includes thermal denaturation information regarding thermal denaturation caused by heat treatment on the biological tissue,
acquire correlation information indicating a preset relationship between an emission intensity and a depth of thermal denaturation from a surface layer in the biological tissue,
determine a presence or absence of the thermal denaturation of a predetermined layer in the biological tissue based on the first image and the second image,
determine a depth of thermal denaturation of the predetermined layer from the surface layer in the biological tissue based on an emission intensity of the fluorescence image and the correlation information, and
output, as support information, depth information regarding the determined depth of thermal denaturation of the predetermined layer from the surface layer in the biological tissue and information indicating the thermal denaturation of the predetermined layer.

2. The medical device according to claim 1, wherein
the layer information includes information regarding a fat layer in the biological tissue.

3. The medical device according to claim 1, wherein
the layer information includes information regarding a layer in the biological tissue.

4. The medical device according to claim 1, wherein
the processor is further configured to determine a presence or absence of the thermal denaturation of a fat layer in the biological tissue based on the first image and the second image.

5. The medical device according to claim 1, wherein
the processor is further configured to
generate a display image with a different display mode for each layer of the biological tissue in which the thermal denaturation has occurred, based on the first image and the second image, and
output the display image.

6. The medical device according to claim 5, wherein
the processor is further configured to
acquire a white light image obtained by imaging the biological tissue irradiated with white light,
generate the display image by superimposing the different display mode for each layer of the biological tissue in which the thermal denaturation has occurred on the white light image, and
output the display image.

7. The medical device according to claim 1, wherein
the processor is further configured to generate a third image including information regarding a muscle layer in the biological tissue.

8. The medical device according to claim 7, wherein
the third image includes information regarding a mucosal layer in the biological tissue.

9. The medical device according to claim 1, wherein
the processor is further configured to
generate a display image in which the thermal denaturation of a layer selected by a user among layers of the biological tissue in which the thermal denaturation has occurred is highlighted, based on the first image and the second image, and
output the display image.

10. A medical system comprising:
a light source device including
a first light source configured to generate first light for acquiring layer information of a biological tissue including a plurality of layers, and
a second light source configured to generate excitation light that excites advanced glycation end products generated by performing heat treatment on the biological tissue,
an imaging device including an imaging element configured to generate an imaging signal by imaging return light or light emitted from the biological tissue irradiated with the first light or the excitation light, and
a medical device including a processor comprising hardware, the processor being configured to
generate a first image including the layer information of the biological tissue including the plurality of layers based on the imaging signal generated by imaging the return light with the imaging element,
generate a second image that is a fluorescence image and includes thermal denaturation information regarding thermal denaturation caused by the heat treatment on the biological tissue based on the imaging signal generated by imaging the emitted light with the imaging element,
acquire correlation information indicating a preset relationship between an emission intensity and a depth of thermal denaturation from a surface layer in the biological tissue,
determine a presence or absence of the thermal denaturation of a predetermined layer in the biological tissue based on the first image and the second image,
determine a depth of thermal denaturation of the predetermined layer from the surface layer in the biological tissue based on an emission intensity of the fluorescence image and the correlation information, and
output, as support information, depth information regarding the determined depth of thermal denaturation of the predetermined layer from the surface layer in the biological tissue and information indicating the thermal denaturation of the predetermined layer.

11. A learning device comprising a processor comprising hardware, the processor being configured to generate a trained model by performing machine learning using training data in which a first image that includes layer information of a biological tissue including a plurality of layers and a second image that is a fluorescence image and includes thermal denaturation information regarding thermal denaturation caused by heat treatment on the biological tissue are input data and depth information regarding a depth of thermal denaturation of a predetermined layer from a surface layer in the biological tissue and information indicating the thermal denaturation of the predetermined layer in the biological tissue are output data.

12. A method of operating a medical device including a processor, the method comprising:
acquiring a first image that includes layer information of a biological tissue including a plurality of layers,
acquiring a second image that is a fluorescence image and includes thermal denaturation information regarding thermal denaturation caused by heat treatment on the biological tissue,
acquiring correlation information indicating a preset relationship between an emission intensity and a depth of thermal denaturation from a surface layer in the biological tissue,
determining a presence or absence of the thermal denaturation of a predetermined layer in the biological tissue based on the first image and the second image,
determining a depth of thermal denaturation of the predetermined layer from the surface layer in the biological tissue based on an emission intensity of the fluorescence image and the correlation information, and
outputting, as support information, depth information regarding the determined depth of thermal denaturation of the predetermined layer from the surface layer in the biological tissue and information indicating the thermal denaturation of the predetermined layer.

13. A non-transitory computer-readable recording medium with an executable program stored thereon, the program causing a processor of a medical device to execute:
acquiring a first image that includes layer information of a biological tissue including a plurality of layers,
acquiring a second image that is a fluorescence image and includes thermal denaturation information regarding thermal denaturation caused by heat treatment on the biological tissue,
acquiring correlation information indicating a preset relationship between an emission intensity and a depth of thermal denaturation from a surface layer in the biological tissue,
determining a presence or absence of the thermal denaturation of a predetermined layer in the biological tissue based on the first image and the second image,
determining a depth of thermal denaturation of the predetermined layer from the surface layer in the biological tissue based on an emission intensity of the fluorescence image and the correlation information, and
outputting, as support information, depth information regarding the determined depth of thermal denaturation of the predetermined layer from the surface layer in the biological tissue and information indicating the thermal denaturation of the predetermined layer.
